(19) United States
(12) Patent Application Publication
(10) Pub. No.: US 2024/0047023 A1
(43) Pub. Date: Feb. 8, 2024

(54) SYSTEM AND METHOD FOR FACILITATING MANAGEMENT OF MEDICAL AND IMMUNIZATION HISTORY OF USERS

(71) Applicant: MyDigiRecords, Overland Park, KS (US)

(72) Inventors: Saroj Gupta, Overland Park, KS (US); Abhimanyu Gupta, Overland Park, KS (US)

(21) Appl. No.: 17/817,369

(22) Filed: Aug. 4, 2022

(51) Int. Cl. G16H 10/60; G16H 40/20; G06F 21/32

(52) U.S. Cl. CPC G16H 10/60; G16H 40/20; G06F 21/32

(57) ABSTRACT

A system and method for facilitating management of medical and immunization history of users is disclosed. The method includes receiving user data, detecting one or more situational parameters, and determining one or more possible immunizations to be received by the user. The method further includes receiving one or more inputs from the one or more user devices to select a desired immunization, transmitting the user data, the one or more situational parameters and the desired immunization in form of a transaction to a healthcare-based blockchain via a decentralized ledger. Further, the healthcare-based blockchain performs one or more blockchain operations including verifying the transaction and adding the verified transaction to the healthcare-based blockchain. Furthermore, the method includes outputting the verified transaction of the healthcare-based blockchain on user interface screen of one or more user devices upon successful authentication of identity of the user.

### FIELD OF INVENTION

Embodiments of the present disclosure relate to data management systems and more particularly relates to a system and a method for facilitating management of medical and immunization history of users.

### BACKGROUND

Generally, it is a responsibility of individuals to maintain immunization information, such as date of receiving the immunizations, types and dates of the immunizations given, and the like. For example, immunizations include Hepatitis A, Hepatitis B, Rotavirus, Diphtheria, Tetanus, Pertussis, Pneumococcal, Inactivated Poliovirus, Influenza, Measles, Mumps, and the like. However, it is very difficult to maintain an accurate record of the immunizations received by the individuals. Conventionally, the individuals often track received immunizations by either keeping a paper trail or remembering when certain immunization took place. However, the paper trail can be easily misplaced or destroyed. Similarly, the individuals may easily forget the immunization information. Thus, an individual may receive the same immunization twice or thrice leading to overdose. There is also a likelihood that the individual may completely miss the immunization. Further, the individuals also rely on a doctor or clinic to maintain records of the received immunizations. However, immunization records are maintained by the doctor or clinic for a limited number of years. Generally, the individuals move, travel, and change health providers due to multiple reasons. Thus, the individuals cannot rely on the doctor or the clinic for their immunization records. Furthermore, there are multiple provider Electronic Medical Records (EMR) systems used by the individuals for accessing the immunization records. However, the provider EMR systems fails to provide access of the immunization records when required. Also, when provider changes EMR or consumer changes the provider, the immunization records are lost. Further, the provider EMR systems also fail to verify the authenticity of the immunization information and securely store the immunization records.

Hence, there is a need for a system and method for facilitating management of medical and immunization history of users, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, a computing system for facilitating management of medical and immunization history of users is disclosed. The computing system includes one or more hardware processors and a memory coupled to the one or more hardware processors. The memory includes a plurality of modules in the form of programmable instructions executable by the one or more hardware processors. The plurality of modules include a user data receiver module configured to receive user data from one or more user devices associated with a user. The user data includes name, age, medical history, one or more medical reports, past surgical history, blood group, one or more past immunization records, one or more emergency contacts, family medical history, social history, allergies of the user, medications the user is currently taking, and medications the user have recently stopped taking. The plurality of modules also include a data detection module configured to detect one or more situational parameters associated with the user by using a location detection technique and predefined medical information upon receiving the user data. The one or more situational parameters include a location, a place and a time of visiting a healthcare facility. Further, the plurality of modules include a data determination module configured to determine one or more possible immunizations to be received by the user from the healthcare facility based on the received user data, provider information and the detected one or more situational parameters. The determined one or more possible immunizations are outputted on user interface screen of the one or more user devices. The plurality of modules further include an input receiver module configured to receive one or more inputs from the one or more user devices to select a desired immunization from the outputted one or more possible immunizations. The desired immunization is an immunization which is one of: received and to be received by the user from the healthcare facility. Also, the plurality of modules include a data transmission module configured to transmit the user data, the one or more situational parameters and the desired immunization in form of a transaction to a healthcare-based blockchain via a decentralized ledger. The healthcare-based blockchain includes a set of blockchain nodes. The set of blockchain nodes of the healthcare-based blockchain correspond to one or more relevant authorities. The healthcare-based blockchain performs one or more blockchain operations for adding the transaction to the healthcare-based blockchain. The one or more blockchain operations. includes verifying the received transaction upon receiving approval from the one or more relevant authorities through a consensus mechanism. Furthermore, the one or more blockchain operations includes adding the verified transaction to the healthcare-based blockchain upon successful verification of the transaction. The plurality of modules include a request receiver module configured to receive a request from the user to access the verified transaction of the healthcare-based blockchain. The verified transaction represents the user data, the one or more situational parameters and the desired immunization. The plurality of modules also include a data authentication module configured to authenticate identity of the user by using one or more authentication techniques upon receiving the request. The one or more authentication techniques include password-based authentication, multi-factor authentication, certificate-based authentication, biometric authentication, and token-based authentication. Further, the plurality of modules also include a data output module configured to output the verified transaction of the healthcare-based blockchain on user interface screen of the one or more user devices upon successfully authenticating the identity of the user.

In accordance with another embodiment of the present disclosure, a method for facilitating management of medical and immunization history of users is disclosed. The method includes receiving user data from one or more user devices associated with a user. The user data includes name, age, medical history, one or more medical reports, past surgical history, blood group, one or more past immunization records, one or more emergency contacts, family medical history, social history, allergies of the user, medications the user is currently taking, and medications the user have recently stopped taking. The method also includes detecting one or more situational parameters associated with the user by using a location detection technique and predefined medical information upon receiving the user data. The one or more situational parameters include a location, a place and a time of visiting a healthcare facility. Further, the method includes determining one or more possible immunizations to be received by the user from the healthcare facility based on the received user data, provider information and the detected one or more situational parameters. The determined one or more possible immunizations are outputted on user interface screen of the one or more user devices. The method further includes receiving one or more inputs from the one or more user devices to select a desired immunization from the outputted one or more possible immunizations. The desired immunization is an immunization which is one of: received and to be received by the user from the healthcare facility. Furthermore, the method includes transmitting the user data, the one or more situational parameters and the desired immunization in form of a transaction to a healthcare-based blockchain via a decentralized ledger. The healthcare-based blockchain includes a set of blockchain nodes. The set of blockchain nodes of the healthcare-based blockchain correspond to one or more relevant authorities. The healthcare-based blockchain performs one or more blockchain operations for adding the transaction to the healthcare-based blockchain. The one or more blockchain operations includes verifying the received transaction upon receiving approval from the one or more relevant authorities through a consensus mechanism The one or more operations also include adding the verified transaction to the healthcare-based blockchain upon successful verification of the transaction. Further, the method includes receiving a request from the user to access the verified transaction of the healthcare-based blockchain. The verified transactions represent the user data, the one or more situational parameters and the desired immunization. The method includes authenticating identity of the user by using one or more authentication techniques upon receiving the request. The one or more authentication techniques include password-based authentication, multi-factor authentication, certificate-based authentication, biometric authentication, and token-based authentication. Furthermore, the method includes outputting the verified transaction of the healthcare-based blockchain on user interface screen of the one or more user devices upon successfully authenticating the identity of the user.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for facilitating management of medical and immunization history of users, in accordance with an embodiment of the present disclosure;

FIG. 2 is a block diagram illustrating the exemplary computing system for facilitating management of medical and immunization history of users, in accordance with an embodiment of the present disclosure;

FIG. 3 is a process flow diagram illustrating an exemplary method for facilitating management of medical and immunization history of users, in accordance with an embodiment of the present disclosure;

FIG. 3A is a process flow diagram illustrating an exemplary method for determining one or more immunization parameters, in accordance with an embodiment of the present disclosure;

FIG. 3B is a process flow diagram illustrating an exemplary method for generating an appointment request to schedule an appointment with a provider, in accordance with an embodiment of the present disclosure;

FIG. 3C is a process flow diagram illustrating an exemplary method for determining one or more possible complications, in accordance with an embodiment of the present disclosure;

FIG. 3D is a process flow diagram illustrating an exemplary method for generating one or more overdosing alerts, in accordance with an embodiment of the present disclosure; and

FIGS. 4A-4E are graphical user interface screens of the exemplary computing system for facilitating management of medical and immunization history of users, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

Although the explanation is limited to a single user. However, it should be understood by the person skilled in the art that the computing system is applied if there are more than one user.

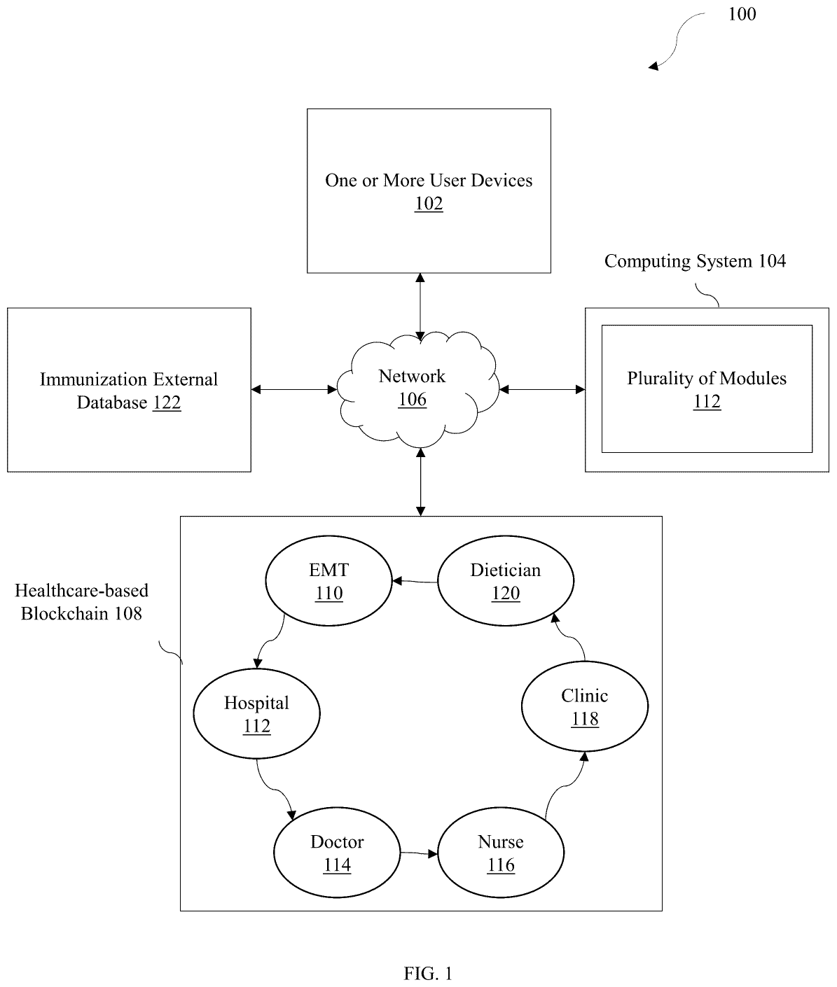

Referring now to the drawings, and more particularly to FIG. 1 through FIG. 4E, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for facilitating management of medical and immunization history of users, in accordance with an embodiment of the present disclosure. According to FIG. 1, the computing environment 100 includes one or more user devices 102 associated with a user communicatively coupled to a computing system 104 via a network 106. The one or more user devices 102 are used to provide user data to the computing system 104. For example, the user data include name, age, medical history, one or more medical reports, past surgical history, blood group, one or more past immunization records, one or more emergency contacts, family medical history, social history, allergies of the user, medications the user is currently taking, medications the user have recently stopped taking, and the like. The one or more user devices 102 are also used by the user to provide one or more inputs to the computing system 104 for selecting a desired immunization from one or more possible immunizations. Further, the one or more user devices 102 are used by the user to receive data associated with a verified transaction of a healthcare-based blockchain 108. In an exemplary embodiment of the present disclosure, the one or more user devices 102 may include a laptop computer, desktop computer, tablet computer, smartphone, wearable device, smart watch, and the like. In an embodiment of the present disclosure, the computing system 104 may be a central server, such as cloud server or a remote server, connected to the healthcare-based blockchain 108. In another embodiment of the present disclosure, the computing system 104 may represent a set of blockchain nodes of the healthcare-based blockchain 108. Further, the network 106 may be internet or any other wireless network.

Further, the computing environment 100 includes the healthcare-based blockchain 108 communicatively coupled to the computing system 104 via the network 106. The healthcare-based blockchain 108 includes a verified transaction representing the user data, one or more situational parameters and a desired immunization. In an embodiment of the present disclosure, the healthcare-based blockchain 108 includes the set of blockchain nodes. The set of blockchain nodes correspond to one or more relevant authorities. In an exemplary embodiment of the present disclosure, the set of blockchain nodes include Emergency Medical Technician (EMT) 110, hospital 112, doctor 114, nurse 116, clinic 118, dietician 120 and the like. In an embodiment of the present disclosure, the set of blockchain nodes approve a transaction representing the user data, the one or more situational parameters and the desired immunization, such the transaction may be verified and added to the healthcare-based blockchain 108.

Further, the computing environment 100 includes an immunization external database 122 communicatively coupled to the computing system 104 via the network 106. In an embodiment of the present disclosure, the immunization external database 122 stores a data representative of immunization. In an exemplary embodiment of the present disclosure, the data representative of immunization includes a list of immunizations, number of doses of each of the list of immunizations required by a person, one or more required intervals between the doses, and the like.

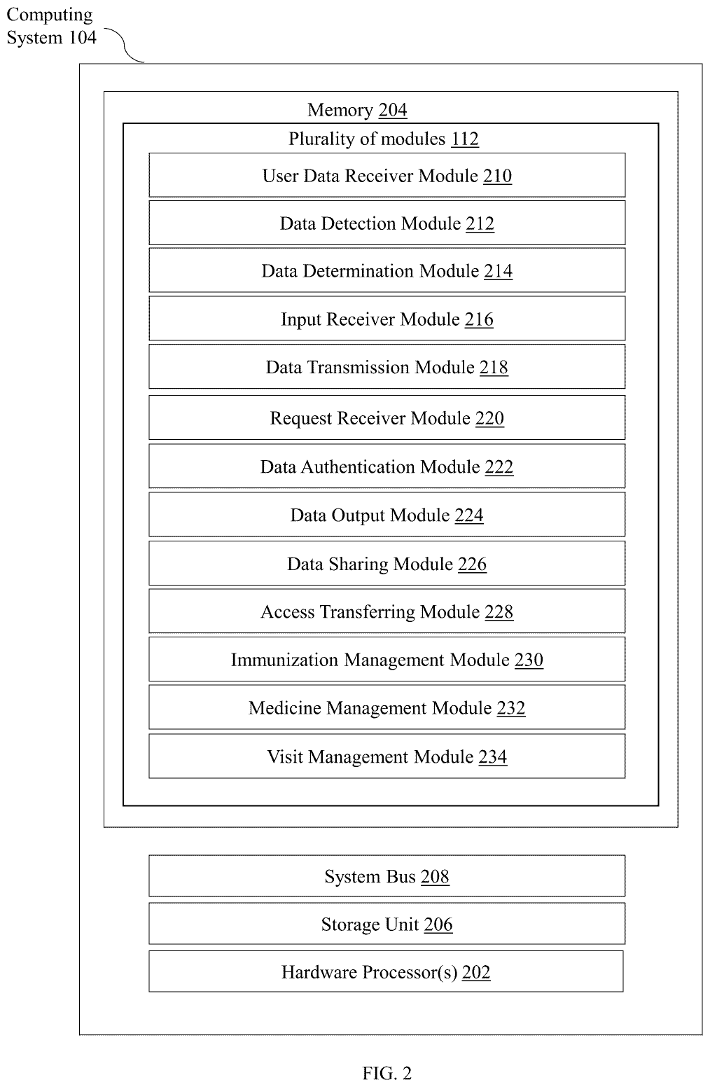

Furthermore, the one or more user devices 102 include a local browser, a mobile application, or a combination thereof. The user may use a web application via the local browser, the mobile application, or a combination thereof to communicate with the computing system 104. In an embodiment of the present disclosure, the computing system 104 includes a plurality of modules 112. Details on the plurality of modules 112 have been elaborated in subsequent paragraphs of the present description with reference to FIG. 2.

In an embodiment of the present disclosure, the computing system 104 is configured to receive the user data from the one or more user devices 102 associated with the user. Further, the computing system 104 detects the one or more situational parameters associated with the user by using a location detection technique and predefined medical information upon receiving the user data. The computing system 104 determines one or more possible immunizations to be received by the user from a healthcare facility based on the received user data, provider information and the detected one or more situational parameters. The computing system 104 receives one or more inputs from the one or more user devices 102 to select a desired immunization from the one or more possible immunizations. Furthermore, the computing system 104 transmits the user data, the one or more situational parameters and the desired immunization in form of a transaction to a healthcare-based blockchain 108 via a decentralized ledger. The healthcare-based blockchain 108 includes a set of blockchain nodes. The set of blockchain nodes of the healthcare-based blockchain 108 correspond to one or more relevant authorities. The healthcare-based blockchain 108 performs one or more blockchain operations for adding the transaction to the healthcare-based blockchain 108. The one or more blockchain operations include verifying the received transaction upon receiving approval from the one or more relevant authorities through a consensus mechanism. The one or more blockchain operations also include adding the verified transaction to the healthcare-based blockchain 108 upon successful verification of the transaction. The computing system 104 receives a request from the user to access the verified transaction of the healthcare-based blockchain 108. Furthermore, the computing system 104 authenticates identity of the user by using one or more authentication techniques upon receiving the request. The computing system 104 outputs the verified transaction of the healthcare-based blockchain 108 on user interface screen of the one or more user devices 102 upon successfully authenticating the identity of the user.

FIG. 2 is a block diagram illustrating the exemplary computing system 104 for facilitating management of medical and immunization history of users, in accordance with an embodiment of the present disclosure. In an embodiment of the present disclosure, the computing system 104 may be a central server connected to the healthcare-based blockchain 108. In another embodiment of the present disclosure, the computing system 104 may represent the set of blockchain nodes of the healthcare-based blockchain 108. Further, the computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 112 in the form of programmable instructions executable by the one or more hardware processors 202. Further, the plurality of modules 112 includes a user data receiver module 210, a data detection module 212, a data determination module 214, an input receiver module 216, a data transmission module 218, a request receiver module 220, a data authentication module 222, a data output module 224, a data sharing module 226, an access transferring module 228, an immunization management module 230, a medicine management module 232, and a visit management module 234.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 112 stored in the form of machine-readable instructions on any of the above-mentioned storage media and may be in communication with and executed by the one or more hardware processors 202.

In an embodiment of the present disclosure, the storage unit 206 may be a cloud storage. The storage unit 206 may store the predefined medical information, the determined one or more possible immunizations, the provider information, the one or more inputs, one or more data-sharing parameters, one or more immunization parameters, a set of prestored identification numbers, one or more locations of one or more providers, a plotted virtual map, one or more transportation options, one or more routes to reach a location of a desired provider, one or more transportation inputs, a set of ingredients of one or more medications, a plurality of ingredients of one or more new medications, one or more possible complications and the like. In another embodiment of the present disclosure, each of the set of blockchain nodes includes the storage unit 306. The storage unit 206 may store the user data, the one or more situational parameters, the desired immunization, and the like.

The user data receiver module 210 is configured to receive the user data from the one or more user devices 102 associated with a user. In an exemplary embodiment of the present disclosure, the user data includes name, age, medical history, one or more medical reports, past surgical history, blood group, one or more past immunization records, one or more emergency contacts, family medical history, social history, allergies of the user, medications the user is currently taking, medications the user have recently stopped taking, and the like. For example, the one or more user devices 102 may include a laptop computer, desktop computer, tablet computer, smartphone, wearable device, smart watch, and the like.

The data detection module 212 is configured to detect the one or more situational parameters associated with the user by using the location detection technique and the predefined medical information upon receiving the user data. For example, the location detection technique may be Global Positioning System (GPS). In an exemplary embodiment of the present disclosure, the one or more situational parameters include a location, a place and a time of visiting a healthcare facility. In an embodiment of the present disclosure, the predefined medical information facilitates in determining which healthcare facility is located at which location. For example, the place may be XYZ health care facility and location may be ABC street.

The data determination module 214 is configured to determine the one or more possible immunizations to be received by the user from the healthcare facility based on the received user data, the provider information and the detected one or more situational parameters. In an embodiment of the present disclosure, the determined one or more possible immunizations are outputted on user interface screen of the one or more user devices 102. For example, the one or more immunizations include Hepatitis A, Hepatitis B, Rotavirus, Diphtheria, Tetanus, Pertussis, Pneumococcal, Inactivated Poliovirus, Influenza, Measles, Mumps, Covid-19 and the like.

The input receiver module 216 is configured to receive the one or more inputs from the one or more user devices 102 to select the desired immunization from the outputted one or more possible immunizations. In an embodiment of the present disclosure, the desired immunization is the immunization which is received or to be received by the user from the healthcare facility.

The data transmission module 218 is configured to transmit the user data, the one or more situational parameters and the desired immunization in form of a transaction to the healthcare-based blockchain 108 via a decentralized ledger. The healthcare-based blockchain 108 includes the set of blockchain nodes. The set of blockchain nodes of the healthcare-based blockchain 108 correspond to the one or more relevant authorities. In an embodiment of the present disclosure, the decentralized ledger is a consensus of replicated, shared, and synchronized digital data geographically spread across multiple sites, countries, or institutions. For example, the one or more relevant authorities may include one or more providers, Emergency Medical Technician (EMT) and the like. In an exemplary embodiment of the present disclosure, the one or more providers include hospitals, doctors, nursing staff, clinics, nursing homes, medical practitioners, nutritionists, dieticians, and the like.

In an embodiment of the present disclosure, the healthcare-based blockchain 108 performs one or more blockchain operations for adding the transaction to the healthcare-based blockchain 108. The one or more blockchain operations includes verifying the received transaction upon receiving approval from the one or more relevant authorities through a consensus mechanism. In an embodiment of the present disclosure, the consensus mechanism is a fault-tolerant mechanism that is used in blockchain systems to achieve a necessary agreement on a single data value or a single state of the network among distributed processes or multi-agent systems. For example, the EMT, the one or more providers or a combination thereof are required to approve the transaction. In an embodiment of the present disclosure, the one or more relevant authorities approve the transaction by providing an identification number, scanning a QR code, selecting approve button on user interface screen of one or more electronic devices associated with the one or more relevant authorities or the like. For example, the one or more electronic devices may include a laptop computer, desktop computer, tablet computer, smartphone, wearable device, smart watch, and the like. In an exemplary embodiment of the present disclosure, the identification number may be National Provider Identifier (NPI), serial number, lot number and the like. For example, both provider and EMT are required to enter their NPI or other number for the application to verify before opening.

Further, the one or more blockchain operations include adding the verified transaction to the healthcare-based blockchain 108 upon successful verification of the transaction. In an embodiment of the present disclosure, the healthcare-based blockchain 108 is different from a traditional database as the healthcare-based blockchain 108 is not a central storage but rather a decentralized, immutable, and secure storage, where information associated with the user data, the one or more situational parameters and the desired immunization may be added in the storage unit 206 upon receiving consensus of the set of nodes. In an embodiment of the present disclosure, the healthcare-based blockchain 108 includes one or more properties, such as the decentralized ledger, smart contracts, security, privacy, decentralization, the consensus mechanism, endorsement, accessibility, and the like. In an embodiment of the present disclosure, the healthcare-based blockchain 108 leverages smart contract logic to store and view the information associated with the user data, the one or more situational parameters and the desired immunization. Further, the information is stored on the decentralized ledger where each of the set of nodes has a copy of the information. To allow the transaction to be committed to the decentralized ledger, there must be consensus between the set of nodes. The computing system 104 greatly improves accessibility for user and healthcare providers to provide consent, transfer data, access data, and keep a record. In an embodiment of the present disclosure, the information may only be modified based on consensus of set of nodes in the healthcare-based blockchain 108.

The request receiver module 220 is configured to receive the request from the user to access the verified transaction of the healthcare-based blockchain 108. In an embodiment of the present disclosure, the verified transactions represent the user data, the one or more situational parameters and the desired immunization.

The data authentication module 222 is configured to authenticate identity of the user by using the one or more authentication techniques upon receiving the request. In an exemplary embodiment of the present disclosure, the one or more authentication techniques include password-based authentication, multi-factor authentication, certificate-based authentication, biometric authentication, token-based authentication, and the like.

The data output module 224 is configured to output the verified transaction of the healthcare-based blockchain 108 on user interface screen of the one or more user devices 102 upon successfully authenticating the identity of the user.

In an embodiment of the present disclosure, the data sharing module 226 is configured to receive one or more identification numbers from the one or more relevant authorities for verifying identity of the one or more relevant authorities. In an exemplary embodiment of the present disclosure, the one or more identification numbers include NPI, serial number, lot number and the like. Further, the data sharing module 226 verifies identity of the one or more relevant authorities by matching the one or more identification numbers with a set of prestored identification numbers. The data sharing module 226 generates one or more data-sharing parameters for sharing the user data, the one or more situational parameters and the desired immunization with the one or more relevant authorities upon verifying identity of the one or more relevant authorities. In an exemplary embodiment of the present disclosure, the one or more data-sharing parameters include one or more Quick Response (QR) codes, one or more certificates and the like. Furthermore, the data sharing module 226 outputs the user data, the one or more situational parameters and the desired immunization on user interface screen of the one or more electronic devices associated with the one or more relevant authorities by using the generated one or more data-sharing parameters. In an embodiment of the present disclosure, the one or more relevant authorities use the one or more data-sharing parameters to receive the user data, the one or more situational parameters and the desired immunization from the user. For example, a QR code is generated in the application for a provider to scan and get the user data. In another example, the one or more user devices 102 display the QR code, such that the EMT may scan the QR code and get all user data in case the user isn't able to provide the user data.

The access transferring module 228 is configured to generate an access code for transferring the user data, the one or more situational parameters and the desired immunization to a new guardian of the user. For example, the access code is a QR code, bar code or the like. Further, the access transferring module 228 transfers the user data, the one or more situational parameters and the desired immunization from original guardian to the new guardian by using the generated access code. In an embodiment of the present disclosure, the new guardian scans the access code to receive the user data, the one or more situational parameters and the desired immunization from the original guardian. In an embodiment of the present disclosure, access of the user data, the one or more situational parameters and the desired immunization is removed for the original guardian. For example, the computing system 104 may accommodate more than one dependent in different accounts. This is useful in case of foster kids, where more than one foster kid is present in a foster family. The foster parents may be able to add the foster kids under one account. If a child moves to a different foster parent, the computing system 104 may allow the original foster parent to create an access code or QR code or another transfer mechanism for that child records to be transferred to a new location and the original parents' access may be removed. For example, the computing system 104 facilitates migrating dependent users from one account to another account.

The immunization management module 230 is configured to obtain a data representative of immunization from an immunization external database 122. In an exemplary embodiment of the present disclosure, the data representative of immunization includes a list of immunizations, number of doses of each of the list of immunizations required by a person, one or more required intervals between the doses, and the like. Further, the immunization management module 230 determines one or more immunization parameters associated with the user based on the user data, the one or more situational parameters, the desired immunization and the obtained data representative of immunization by using an immunization management-based Artificial Intelligence (AI) model. In an exemplary embodiment of the present disclosure, the one or more immunization parameters include one or more necessary immunizations, number of doses of each of the one or more necessary immunizations, time of receiving the one or more doses for each of the one or more necessary immunizations, and the like. The immunization management module 230 detects the one or more providers of the one or more necessary immunizations and one or more locations of the one or more providers in vicinity of the user based on current location of the user, the determined one or more immunization parameters and predefined provider information. In an exemplary embodiment of the present disclosure, the predefined provider information includes information related to the one or more providers and one or more immunizations provided by each of the one or more providers. Furthermore, the immunization management module 230 plots the detected one or more locations of the detected one or more providers on a virtual map. The immunization management module 230 outputs the determined one or more immunization parameters, the detected one or more providers, the one or more locations of the one or more providers and the plotted virtual map on user interface screen of the one or more user devices 102.

Further, the immunization management module 230 obtains availability information associated with the detected one or more providers from an external provider database. In an exemplary embodiment of the present disclosure, the availability information includes one or more available slots for each of the one or more providers, a type of insurance plan accepted by the one or more providers, working days, working hours of each of the one or more providers and the like. The immunization management module 230 receives one or more inputs from the user to select a desired provider from the detected one or more providers and a desired time to visit the desired provider upon obtaining the availability information. For example, the user may visit the provider who accepts an insurance plan of the user. Furthermore, the immunization management module 230 generates an appointment request to schedule an appointment with the desired provider at the desired time based on the obtained availability information and the received one or more inputs. In an embodiment of the present disclosure, the appointment is scheduled with the desired provider when the generated appointment request is approved by the desired provider. The immunization management module 230 detects one or more transportation options and one or more routes to reach a location of the desired provider based on the current location of the user, the location of the desired provider and a predefined location information by using a transportation detection-based AI model. The predefined location information is a geolocation information used to detect the one or more transportation options and the one or more routes. For example, the one or more transportation options may be bus, train, and the like. In an embodiment of the present disclosure, the detected one or more transportation options and the detected one or more routes are outputted on user interface screen of the one or more user devices 102. The immunization management module 230 receives one or more transportation inputs from the user to purchase one or more tickets of the one or more transportation options to reach the location of the desired provider upon detecting the one or more transportation options and the one or more routes. In an exemplary embodiment of the present disclosure, the one or more transportation inputs include number of tickets, name of the user, a payment mode, a desired route, a desired transportation option, one or more seat numbers, and the like. In another embodiment of the present disclosure, the one or more transportation inputs may be used to book a cab service, such as Uber®, Ola® and the like.

The medicine management module 232 obtains medicine information associated with the user from the healthcare-based blockchain 108. In an embodiment of the present disclosure, the medicine information includes one or more medications the user is currently taking. Further, the medicine management module 232 detects a set of ingredients of the one or more medications based on the obtained medicine information and a predefined ingredient information. The predefined ingredient information facilitates in determining ingredients of a medicine. Further, the medicine management module 232 receives one or more new medications for the user from one or more medical professionals. The medicine management module 232 detects a plurality of ingredients of the received one or more new medications based on the predefined ingredient information. Furthermore, the medicine management module 232 determines one or more possible complications corresponding to the one or more new medications based on the user data, the obtained medicine information, the detected set of ingredients, the detected plurality of ingredients and a predefined complication information by using a medication management-based AI model. For example, the predefined complication information facilitates in determining which two ingredients in combination may cause complications to the user. In an exemplary embodiment of the present disclosure, the one or more complications include slowed breathing, blood thinning, poisoning, skin rashes, increase in blood pressure and the like. The medicine management module 232 outputs the determined one or more possible complications on user interface screen of the one or more user devices 102 and the one or more provider devices associated with one or more providers.

Further, the medicine management module 232 determines if the one or more new medications are similar to the one or more medications based on the detected set of ingredients of the one or more medications and the detected plurality of ingredients of the received one or more new medications by using the medication management-based AI model. The medicine management module 232 generates one or more overdosing alerts upon determining that the one or more new medications are similar to the one or more medications. Furthermore, the medicine management module 232 outputs the generated one or more overdosing alerts on user interface screen of the one or more user devices 102 and the one or more provider devices. For example, if a medicine is already being taken and the prescriber prescribes another medicine but different name, a notification is sent out to the provider and the user or others as needed.

In an exemplary embodiment of the present disclosure, the immunization management-based AI model and the medication management-based AI model may be deep learning models, such as Region-Based Convolutional Neural Network (RCNN), Convolutional Neural Network (CNN), You Only Look Once (YOLO) for object detection, and the like. In an embodiment of the present disclosure, tensor flow and PyTorch are used to develop AI models.

Furthermore, the medicine management module 232 obtains dose information associated with the user from the healthcare-based blockchain 108, the user, a medical prescription, the one or more providers, or a combination thereof. In an embodiment of the present disclosure, the dose information is obtained by adding the dose information manually, scanning the medical prescription, receiving the dose information from the provider via a dashboard, or the like. For example, the dose information includes one or more prescribed medications prescribed for the user, a number of required doses of the one or more prescribed medications, one or more prescribed immunizations, a number of required doses of the one or more prescribed immunizations, a required time of taking each of the required doses, a required time for performing one or more medical exercise therapies, a time duration for which the user is required to take the one or more prescribed medications, and the like. Further, the medicine management module 232 generates one or more medicine alerts for the user based on the obtained dose information. In an exemplary embodiment of the present disclosure, the one or more medicine alerts are generated for reminding the user to take the one or more prescribed medications at the required time, visit one or more medical facilities to take the one or more prescribed immunizations at the required time, stop taking the one or more prescribed medications after expiry of the time duration, perform the one or more medical exercise therapies at the required time and the like. Furthermore, the medicine management module 232 outputs the generated one or more medicine alerts on user interface screen of the one or more user devices 102. For example, the computing system 104 keeps track of how long to take a medicine and alerts the user at the end of the timeframe to not take the medicine again. In an embodiment of the present disclosure, the timeframe may be calculated from the day on which first dose is taken. (supportive)

In an embodiment of the present disclosure, when the user is not able to speak in English, the user may text the provider in their native language using the application. Further, the computing system 104 may translate the text in native language to English and vice versa.

The visit management module 234 obtains the provider information associated with one or more providers based on the detected one or more situational parameters, the one or more identification numbers, a provider's directory, a calendar entry of the user with the provider's name and address, or a combination thereof. In an embodiment of the present disclosure, the provider information may be obtained from a provider external database. For example, the provider information includes name, address, a medical care provided by the one or more providers, and the like. In an embodiment of the present disclosure, the medical care includes injecting one or more doses of immunizations, prescribing one or more medications and the like. Further, the visit management module 234 determines if the user already visited one or more medical facilities to receive a similar medical care as provided by the one or more providers based on the obtained provider information and by accessing the verified transaction of the healthcare-based blockchain 108. The visit management module 234 generates one or more overdosing alerts upon determining that the user already visited the one or more medical facilities to receive the similar medical care as provided by the one or more providers. Furthermore, the visit management module 234 outputs the generated one or more overdosing alerts on user interface screen of the one or more user devices 102 and the one or more provider devices associated with the one or more providers. For example, the computing system 104 keeps track of where and when a medicine and immunization was prescribed, taken or a combination thereof. This may help in opioid overdose, medical marijuana overdose and the like. For example, the computing system 104 may track what medical facilities a user is visiting and obtains the provider information in case a provider enters their NPI number so to match quickly. The medical facilities may be tracked by using a geolocation and accessing the provider's directory. In an embodiment of the present disclosure, If the user has a calendar entry with the provider's name and address, the calendar entry may also be used for the tracking. Further, the tracking of the medical facilities may help to avoid opioid overdose and the provider may be notified of the user's previous visits to another facility.

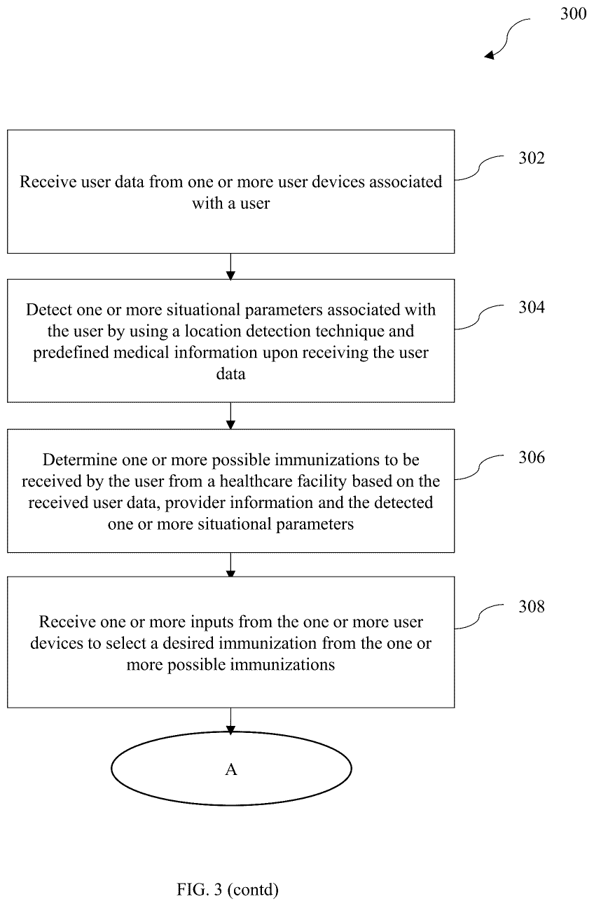

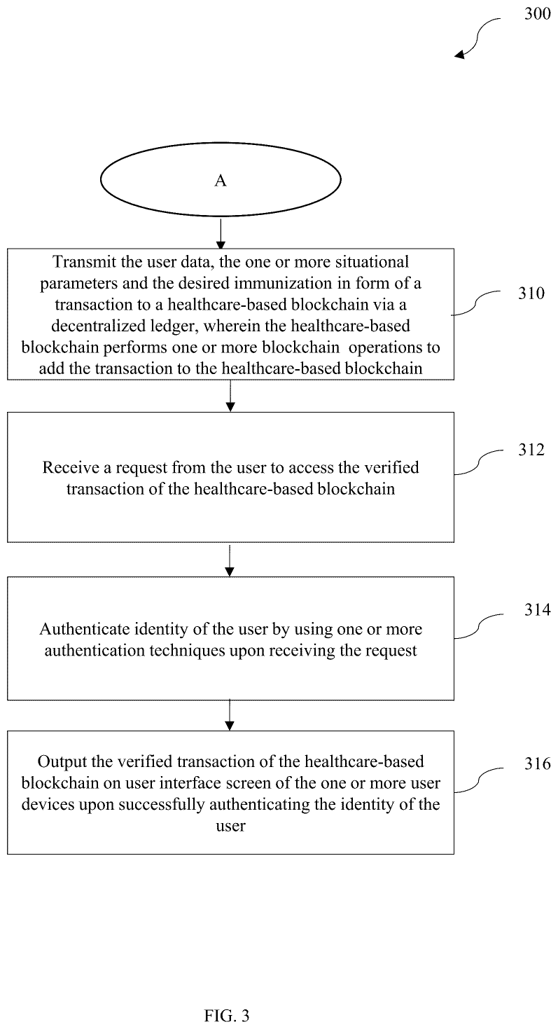

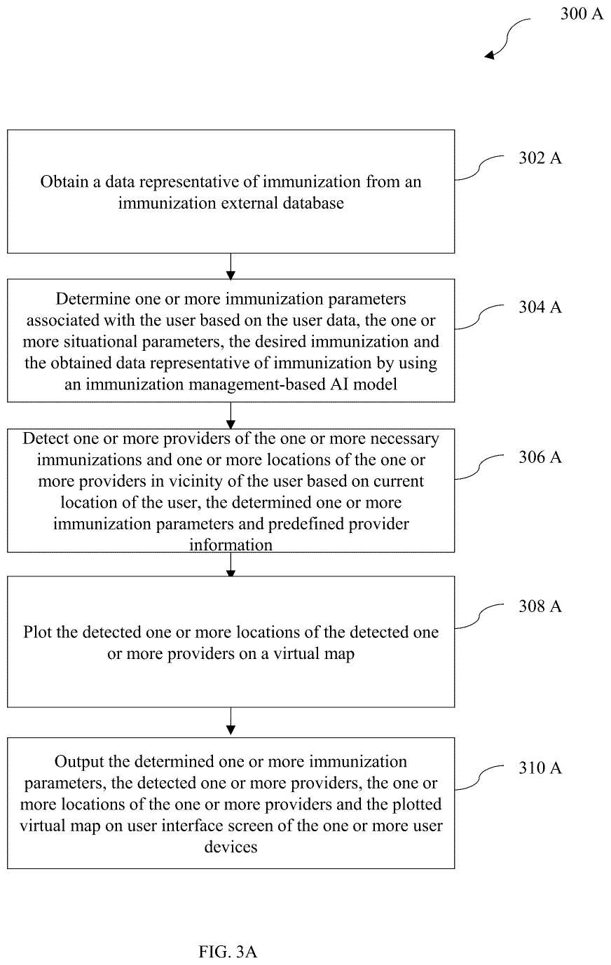

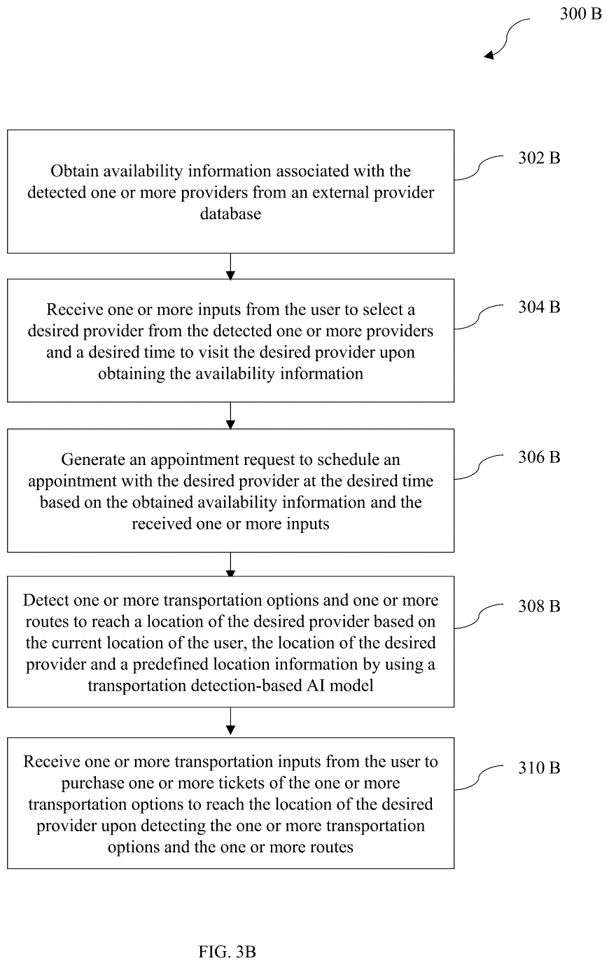

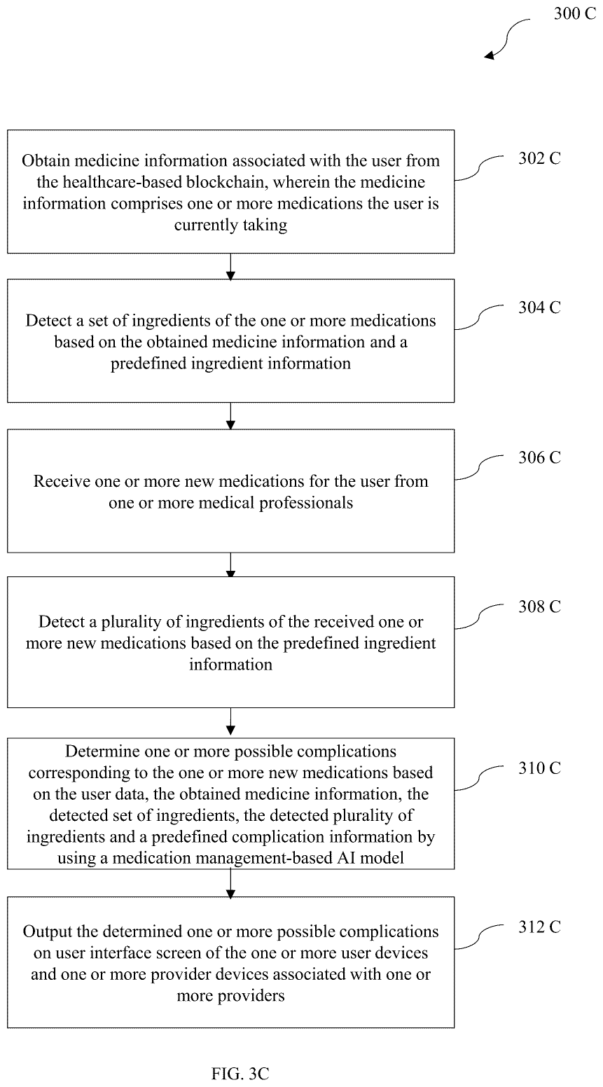

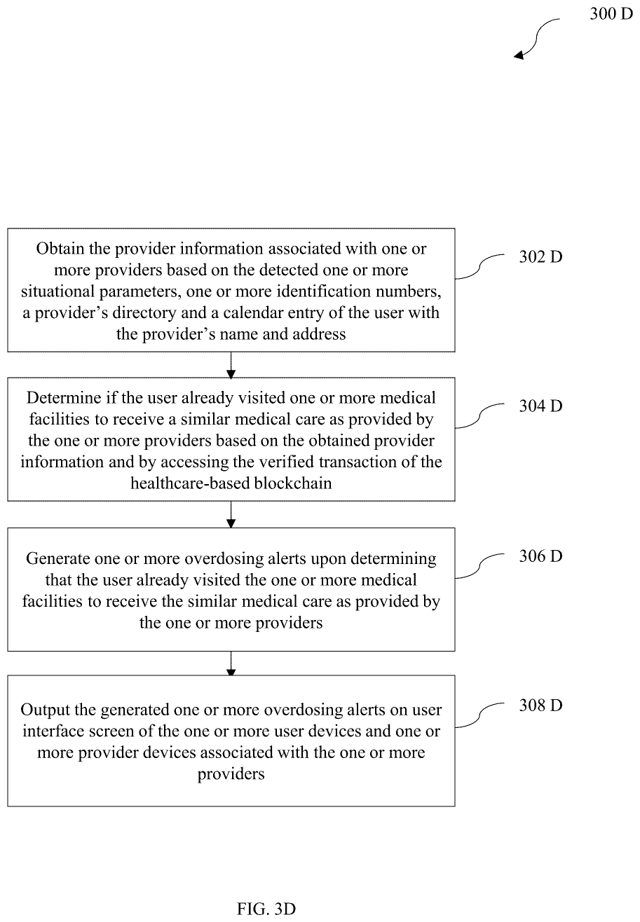

FIG. 3 is a process flow diagram illustrating an exemplary method for facilitating management of medical and immunization history of users, in accordance with an embodiment of the present disclosure. At step 302, user data is received from one or more user devices 102 associated with a user. In an exemplary embodiment of the present disclosure, the user data includes name, age, medical history, one or more medical reports, past surgical history, blood group, one or more past immunization records, one or more emergency contacts, family medical history, social history, allergies of the user, medications the user is currently taking, medications the user have recently stopped taking, and the like. For example, the one or more user devices 102 may include a laptop computer, desktop computer, tablet computer, smartphone, wearable device, smart watch, and the like.

At step 304, one or more situational parameters associated with the user are detected by using a location detection technique and predefined medical information upon receiving the user data. For example, the location detection technique may be GPS. In an exemplary embodiment of the present disclosure, the one or more situational parameters include a location, a place and a time of visiting a healthcare facility. In an embodiment of the present disclosure, the predefined medical information facilitates in determining which healthcare facility is located at which location.

At step 306, one or more possible immunizations to be received by the user are determined from the healthcare facility based on the received user data, provider information and the detected one or more situational parameters. In an embodiment of the present disclosure, the determined one or more possible immunizations are outputted on user interface screen of the one or more user devices 102. For example, the one or more immunizations include Hepatitis A, Hepatitis B, Rotavirus, Diphtheria, Tetanus, Pertussis, Pneumococcal, Inactivated Poliovirus, Influenza, Measles, Mumps, Covid-19 and the like.

At step 308, one or more inputs are received from the one or more user devices 102 to select a desired immunization from the outputted one or more possible immunizations. In an embodiment of the present disclosure, the desired immunization is the immunization which is received or to be received by the user from the healthcare facility.

At step 310, the user data, the one or more situational parameters and the desired immunization in form of a transaction are transmitted to the healthcare-based blockchain 108 via a decentralized ledger. The healthcare-based blockchain 108 includes the set of blockchain nodes. The set of blockchain nodes of the healthcare-based blockchain 108 correspond to the one or more relevant authorities. In an embodiment of the present disclosure, the decentralized ledger is a consensus of replicated, shared, and synchronized digital data geographically spread across multiple sites, countries, or institutions. For example, the one or more relevant authorities may include one or more providers, EMT and the like. In an exemplary embodiment of the present disclosure, the one or more providers include hospitals, doctors, nursing staff, clinics, nursing homes, medical practitioners, nutritionists, dieticians, and the like.

In an embodiment of the present disclosure, the healthcare-based blockchain 108 performs one or more blockchain operations for adding the transaction to the healthcare-based blockchain 108. The one or more blockchain operations includes verifying the transaction upon receiving approval from the one or more relevant authorities through a consensus mechanism. In an embodiment of the present disclosure, the consensus mechanism is a fault-tolerant mechanism that is used in blockchain systems to achieve a necessary agreement on a single data value or a single state of the network among distributed processes or multi-agent systems. For example, the EMT, the one or more providers or a combination thereof are required to approve the transaction. In an embodiment of the present disclosure, the one or more relevant authorities approve the transaction by providing an identification number, scanning a QR code, selecting approve button on user interface screen of one or more electronic devices associated with the one or more relevant authorities or the like. For example, the one or more electronic devices may include a laptop computer, desktop computer, tablet computer, smartphone, wearable device, smart watch, and the like. In an exemplary embodiment of the present disclosure, the identification number may be National Provider Identifier (NPI), serial number, lot number and the like. For example, both provider and EMT are required to enter their NPI or other number for the application to verify before opening.

Further, the one or more blockchain operations include adding the verified transaction to the healthcare-based blockchain 108 upon successful verification of the transaction.

In an embodiment of the present disclosure, the healthcare-based blockchain 108 is different from a traditional database as the healthcare-based blockchain 108 is not a central storage but rather a decentralized, immutable, and secure storage, where information associated with the user data, the one or more situational parameters and the desired immunization may be added in the storage unit 206 upon receiving consensus of the set of nodes. In an embodiment of the present disclosure, the healthcare-based blockchain 108 includes one or more properties, such as the decentralized ledger, smart contracts, security, privacy, decentralization, the consensus mechanism, endorsement, accessibility, and the like. In an embodiment of the present disclosure, the healthcare-based blockchain 108 leverages smart contract logic to store and view the information associated with the user data, the one or more situational parameters and the desired immunization. Further, the information is stored on the decentralized ledger where each of the set of nodes has a copy of the information. To allow the transaction to be committed to the decentralized ledger, there must be consensus between the set of nodes. The computing system 104 greatly improves accessibility for user and healthcare providers to provide consent, transfer data, access data, and keep a record. In an embodiment of the present disclosure, the information may only be modified based on consensus of set of nodes in the healthcare-based blockchain 108.

At step 312, a request is received from the user to access the verified transaction of the healthcare-based blockchain 108. In an embodiment of the present disclosure, the verified transactions represent the user data, the one or more situational parameters and the desired immunization.

At step 314, identity of the user is authenticated by using one or more authentication techniques upon receiving the request. In an exemplary embodiment of the present disclosure, the one or more authentication techniques include password-based authentication, multi-factor authentication, certificate-based authentication, biometric authentication, token-based authentication, and the like.

At step 316, the verified transaction of the healthcare-based blockchain 108 is outputted on user interface screen of the one or more user devices 102 upon successfully authenticating the identity of the user.

In an embodiment of the present disclosure, the method 300 includes receiving one or more identification numbers from the one or more relevant authorities for verifying identity of the one or more relevant authorities. In an exemplary embodiment of the present disclosure, the one or more identification numbers include NPI, serial number, lot number and the like. Further, the method 300 includes verifying identity of the one or more relevant authorities by matching the one or more identification numbers with a set of prestored identification numbers. The method 300 includes generating one or more data-sharing parameters for sharing the user data, the one or more situational parameters and the desired immunization with the one or more relevant authorities upon verifying identity of the one or more relevant authorities. In an exemplary embodiment of the present disclosure, the one or more data-sharing parameters include one or more Quick Response (QR) codes, one or more certificates and the like. Furthermore, the method 300 includes outputting the user data, the one or more situational parameters and the desired immunization on user interface screen of the one or more electronic devices associated with the one or more relevant authorities by using the generated one or more data-sharing parameters. In an embodiment of the present disclosure, the one or more relevant authorities use the one or more data-sharing parameters to receive the user data, the one or more situational parameters and the desired immunization from the user. For example, a QR code is generated in the application for a provider to scan and get the user data. In another example, the one or more user devices 102 display the QR code, such that the EMT may scan the QR code and get all user data in case the user isn't able to provide the user data.

Further, the method 300 includes generating an access code for transferring the user data, the one or more situational parameters and the desired immunization to a new guardian of the user. For example, the access code is a QR code, bar code or the like. Further, the method 300 includes transferring the user data, the one or more situational parameters and the desired immunization from original guardian to the new guardian by using the generated access code. In an embodiment of the present disclosure, the new guardian scans the access code to receive the user data, the one or more situational parameters and the desired immunization from the original guardian. In an embodiment of the present disclosure, access of the user data, the one or more situational parameters and the desired immunization is removed for the original guardian. For example, the computing system 104 may accommodate more than one dependent in different accounts. This is useful in case of foster kids, where more than one foster kid is present in a foster family. The foster parents may be able to add the foster kids under one account. If a child moves to a different foster parent, the computing system 104 may allow the original foster parent to create an access code or QR code or another transfer mechanism for that child records to be transferred to a new location and the original parents' access may be removed. For example, the computing system 104 facilitates migrating dependent users from one account to another account.

FIG. 3A is a process flow diagram illustrating an exemplary method for determining one or more immunization parameters, in accordance with an embodiment of the present disclosure. At step 302 A, the method 300 A includes obtaining a data representative of immunization from an immunization external database 122. In an exemplary embodiment of the present disclosure, the data representative of immunization includes a list of immunizations, number of doses of each of the list of immunizations required by a person, one or more required intervals between the doses, and the like. At step 304 A, the method 300 A includes determining one or more immunization parameters associated with the user based on the user data, the one or more situational parameters, the desired immunization and the obtained data representative of immunization by using an immunization management-based Artificial Intelligence (AI) model. In an exemplary embodiment of the present disclosure, the one or more immunization parameters include one or more necessary immunizations, number of doses of each of the one or more necessary immunizations, time of receiving the one or more doses for each of the one or more necessary immunizations, and the like. At step 306 A, the method 300 A includes detecting the one or more providers of the one or more necessary immunizations and one or more locations of the one or more providers in vicinity of the user based on current location of the user, the determined one or more immunization parameters and predefined provider information. In an exemplary embodiment of the present disclosure, the predefined provider information includes information related to the one or more providers and one or more immunizations provided by each of the one or more providers. At step 308 A, the method 300 A includes plotting the detected one or more locations of the detected one or more providers on a virtual map. At step 310 A, the method 300 A includes outputting the determined one or more immunization parameters, the detected one or more providers, the one or more locations of the one or more providers and the plotted virtual map on user interface screen of the one or more user devices 102.

FIG. 3B is a process flow diagram illustrating an exemplary method for generating an appointment request to schedule an appointment with a provider, in accordance with an embodiment of the present disclosure. At step 302 B, the method 300 B includes obtaining availability information associated with the detected one or more providers from an external provider database. In an exemplary embodiment of the present disclosure, the availability information includes one or more available slots for each of the one or more providers, a type of insurance plan accepted by the one or more providers, working days, working hours of each of the one or more providers and the like. At step 304 B, the method 300 B includes receiving one or more inputs from the user to select a desired provider from the detected one or more providers and a desired time to visit the desired provider upon obtaining the availability information. For example, the user may visit the provider who accepts an insurance plan of the user. At step 306 B, the method 300 B includes generating an appointment request to schedule an appointment with the desired provider at the desired time based on the obtained availability information and the received one or more inputs. In an embodiment of the present disclosure, the appointment is scheduled with the desired provider when the generated appointment request is approved by the desired provider. At step 308 B, the method 300 B includes detecting one or more transportation options and one or more routes to reach a location of the desired provider based on the current location of the user, the location of the desired provider and a predefined location information by using a transportation detection-based AI model. The predefined location information is a geolocation information used to detect the one or more transportation options and the one or more routes. For example, the one or more transportation options may be bus, train, and the like. In an embodiment of the present disclosure, the detected one or more transportation options and the detected one or more routes are outputted on user interface screen of the one or more user devices 102. At step 310 B, the method 300 B includes receiving one or more transportation inputs from the user to purchase one or more tickets of the one or more transportation options to reach the location of the desired provider upon detecting the one or more transportation options and the one or more routes. In an exemplary embodiment of the present disclosure, the one or more transportation inputs include number of tickets, name of the user, a payment mode, a desired route, a desired transportation option, one or more seat numbers, and the like. In another embodiment of the present disclosure, the one or more transportation inputs may be used to book a cab service, such as Uber®, Ola® and the like.

FIG. 3C is a process flow diagram illustrating an exemplary method for determining one or more possible complications, in accordance with an embodiment of the present disclosure. At step 302 C, the method 300 C includes obtaining medicine information associated with the user from the healthcare-based blockchain 108. In an embodiment of the present disclosure, the medicine information includes one or more medications the user is currently taking. At step 304 C, the method 300 C includes detecting a set of ingredients of the one or more medications based on the obtained medicine information and a predefined ingredient information. The predefined ingredient information facilitates in determining ingredients of a medicine. At step 306 C, the method 300 C includes receiving one or more new medications for the user from one or more medical professionals. At step 308 C, the method 300 C includes detecting a plurality of ingredients of the received one or more new medications based on the predefined ingredient information. At step 310 C, the method 300 C includes determining one or more possible complications corresponding to the one or more new medications based on the user data, the obtained medicine information, the detected set of ingredients, the detected plurality of ingredients and a predefined complication information by using a medication management-based AI model. For example, the predefined complication information facilitates in determining which two ingredients in combination may cause complications to the user. In an exemplary embodiment of the present disclosure, the one or more complications include slowed breathing, blood thinning, poisoning, skin rashes, increase in blood pressure and the like. At step 312 C, the method 300 C includes outputting the determined one or more possible complications on user interface screen of the one or more user devices 102 and the one or more provider devices associated with one or more providers.

Further, the method 300 C includes determining if the one or more new medications are similar to the one or more medications based on the detected set of ingredients of the one or more medications and the detected plurality of ingredients of the received one or more new medications by using the medication management-based AI model. The method 300 C includes generating one or more overdosing alerts upon determining that the one or more new medications are similar to the one or more medications. Furthermore, the method 300 C includes outputting the generated one or more overdosing alerts on user interface screen of the one or more user devices 102 and the one or more provider devices. For example, if a medicine is already being taken and the prescriber prescribes another medicine but different name, a notification is sent out to the provider and the user or others as needed.

In an exemplary embodiment of the present disclosure, the immunization management-based AI model and the medication management-based AI model may be deep learning models, such as RCNN, CNN, YOLO for object detection, and the like. In an embodiment of the present disclosure, tensor flow and PyTorch are used to develop AI models.

Furthermore, the method 300 C includes obtaining dose information associated with the user from the healthcare-based blockchain 108, the user, a medical prescription, the one or more providers, or a combination thereof. In an embodiment of the present disclosure, the dose information is obtained by adding the dose information manually, scanning the medical prescription, receiving the dose information from the provider via a dashboard, or the like. For example, the dose information includes one or more prescribed medications prescribed for the user, a number of required doses of the one or more prescribed medications, one or more prescribed immunizations, a number of required doses of the one or more prescribed immunizations, a required time of taking each of the required doses, a required time for performing one or more medical exercise therapies, a time duration for which the user is required to take the one or more prescribed medications, and the like. Further, the method 300 C includes generating one or more medicine alerts for the user based on the obtained dose information. In an exemplary embodiment of the present disclosure, the one or more medicine alerts are generated for reminding the user to take the one or more prescribed medications at the required time, visit one or more medical facilities to take the one or more prescribed immunizations at the required time, stop taking the one or more prescribed medications after expiry of the time duration, perform the one or more medical exercise therapies at the required time and the like. Furthermore, the method 300 C includes outputting the generated one or more medicine alerts on user interface screen of the one or more user devices 102. For example, the computing system 104 keeps track of how long to take a medicine and alerts the user at the end of the timeframe to not take the medicine again. In an embodiment of the present disclosure, the timeframe may be calculated from the day on which first dose is taken.

In an embodiment of the present disclosure, when the user is not able to speak in English, the user may text the provider in their native language using the application. Further, the computing system 104 may translate the text in native language to English and vice versa.

FIG. 3D is a process flow diagram illustrating an exemplary method for generating one or more overdosing alerts, in accordance with an embodiment of the present disclosure. At step 302 D, the method 300 D includes obtaining the provider information associated with one or more providers based on the detected one or more situational parameters, the one or more identification numbers, a provider's directory, a calendar entry of the user with the provider's name and address, or a combination thereof. In an embodiment of the present disclosure, the provider information may be obtained from a provider external database. For example, the provider information includes name, address, a medical care provided by the one or more providers, and the like. In an embodiment of the present disclosure, the medical care includes injecting one or more doses of immunizations, prescribing one or more medications and the like. At step 304 D, the method 300 D includes determining if the user already visited one or more medical facilities to receive a similar medical care as provided by the one or more providers based on the obtained provider information and by accessing the verified transaction of the healthcare-based blockchain 108. At step 306 D, the method 300 D includes generating one or more overdosing alerts upon determining that the user already visited the one or more medical facilities to receive the similar medical care as provided by the one or more providers. At step 308 D, the method 300 D includes outputting the generated one or more overdosing alerts on user interface screen of the one or more user devices 102 and the one or more provider devices associated with the one or more providers. For example, the computing system 104 keeps track of where and when a medicine and immunization was prescribed, taken or a combination thereof. This may help in opioid overdose, medical marijuana overdose and the like. For example, the computing system 104 may track what medical facilities a user is visiting and obtains the provider information in case a provider enters their NPI number so to match quickly. The medical facilities may be tracked by using a geolocation and accessing the provider's directory. In an embodiment of the present disclosure, If the user has a calendar entry with the provider's name and address, the calendar entry may also be used for the tracking. Further, the tracking of the medical facilities may help to avoid opioid overdose and the provider may be notified of the user's previous visits to another facility.

The method 300 may be implemented in any suitable hardware, software, firmware, or combination thereof.

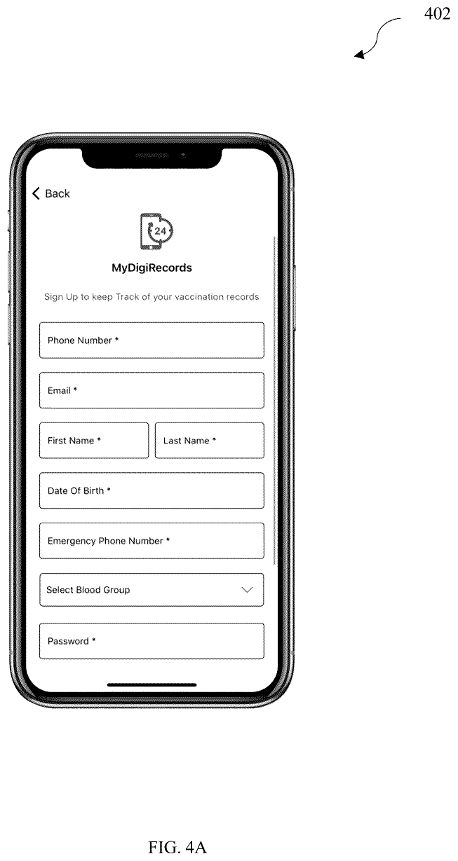

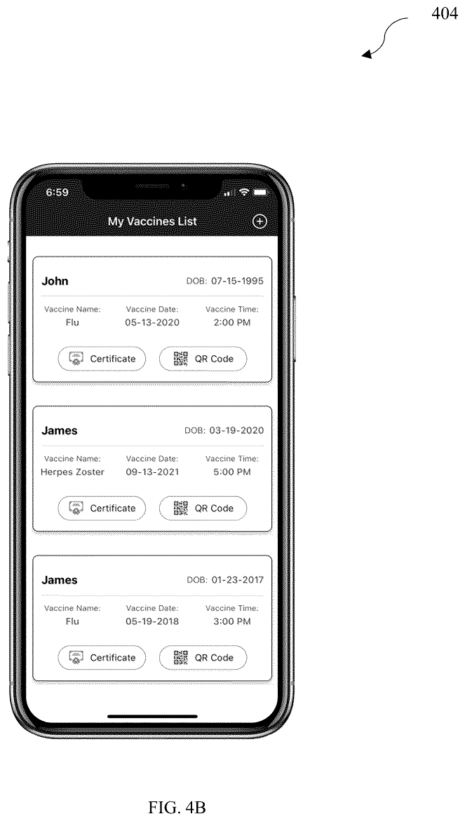

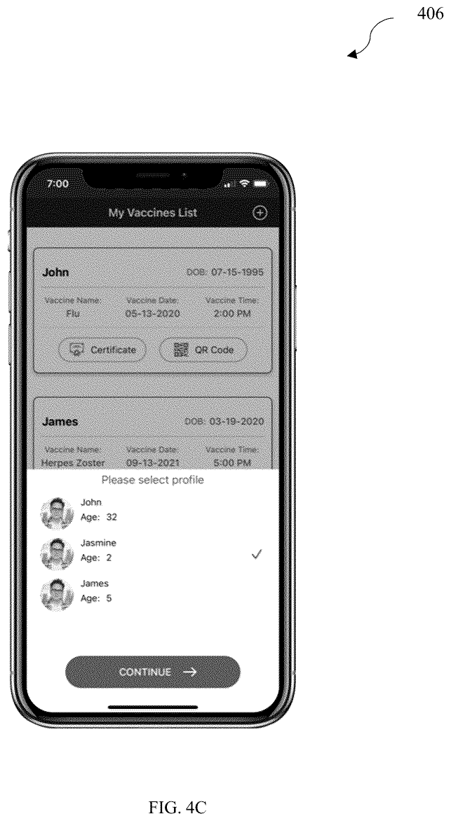

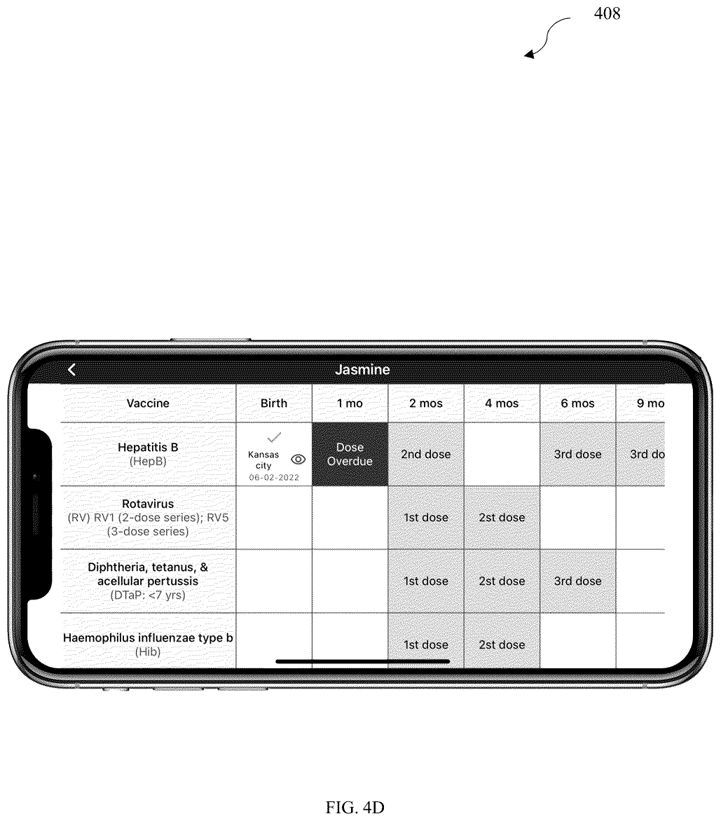

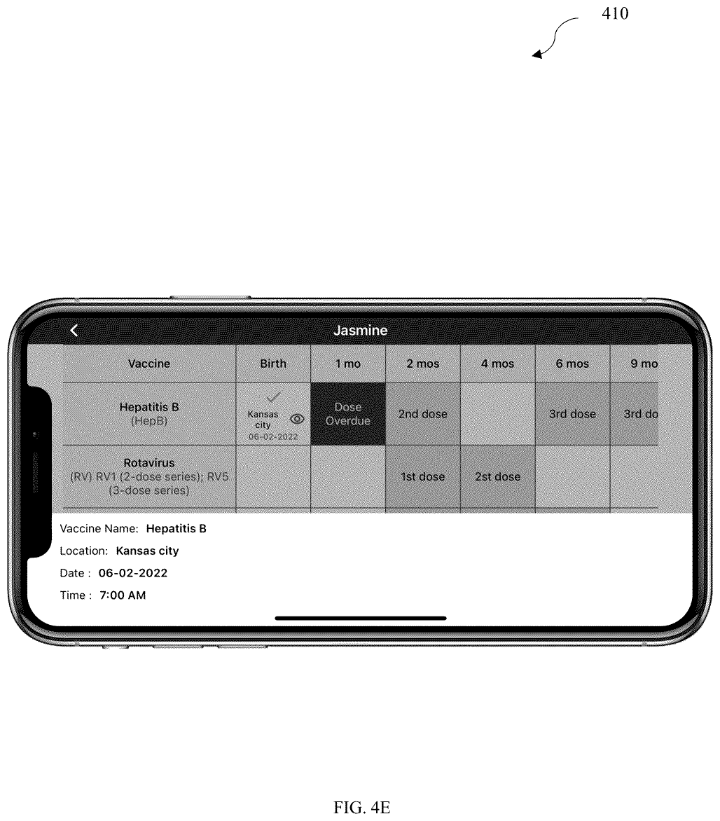

FIGS. 4A-4E are graphical user interface screens of the exemplary computing system for facilitating management of medical and immunization history of users, in accordance with an embodiment of the present disclosure. A graphical user interface screen 402 of FIG. 4A is used to register the user by receiving the user data from the user. For example, the user data may be phone number, email, first name, last name, date of birth, emergency contact number, blood group of the user and password. Further, a graphical user interface screen 404 of FIG. 4B displays the immunization list of users including immunization information, such as immunization name, immunization date, immunization time and the like. The immunization information may be shared by using the QR code and the certificate. A graphical user interface screen 406 of FIG. 4C is used to by the user for selecting a profile from multiple saved profiles. Furthermore, a graphical user interface screen 408 of FIG. 4D displays immunization schedule of the user. The graphical user interface screen 408 also displays which immunization is overdue and which immunizations are already received by the user. Further, a graphical user interface screen 408 of FIG. 4E displays the immunization information of the immunization which is overdue including immunization name, location, date, time and the like of the immunization to be received by the user.

Thus, various embodiments of the present computing system 104 provide a solution to facilitate facilitating management of medical and immunization history of users. The computing system 104 enables the user to have their immunization history with them digitally on an application whenever they desire. Thus, the computing system 104 helps the user to avoid over and under immunization causing serious illness. Further, the computing system 104 also allows the states to get proper records of total number of immunizations provided on time. In an embodiment of the present disclosure, the computing system 104 allows the user to enter their immunization records with the help of provider NPI, such that the user may access the immunization records at later stage. The computing system 104 includes a dashboard for providers for emergency immunizations, such as Covid-19 and the immunization records may be populated on the application. Furthermore, the computing system 104 notifies the user of any upcoming immunization need and shows a map of providers in the vicinity that provide the services. The computing system 104 also enables a user to book a time on the provider's calendar. In an embodiment of the present disclosure, when a medicine is entered, the computing system 104 keeps track of all the ingredients and if another medicine is prescribed that can cause conflict, a notification is sent to both the provider, the user and the caretaker or others as required. Further, if a medicine is already being taken and the prescriber prescribes another medicine but different name, a notification is sent out to the provider and the user or others as needed. The computing system 104 provides updates on ingredients of medicines conflicting with each other. Furthermore, the computing system 104 also provides updates if that a medicine is already being taken by the user with another name. The computing system 104 verifies the authenticity of usage data and securely store the usage data in the healthcare-based blockchain 108. In an embodiment of the present disclosure, the computing system 104 is having a dashboard for providers to record immunizations taken by the user from birth to death and update the immunization records stored in the healthcare-based blockchain 108. The computing system 104 may accommodate more than one dependent person in different accounts, where the dependent accounts are formed and can be transferred to or from different accounts due to marriage, change in foster parents and the like. Further, the computing system 104 uses the healthcare-based blockchain 108 that keeps track of where and when a medicine and immunization was prescribed and/or taken. This could help in avoiding an overdose or over-prescribing drugs. Furthermore, the computing system 104 uses the healthcare-based blockchain 108 to keeps track of how long to take the medicine, sends reminders to take medicines, and alerts the user at the end of the timeframe to not take the medicine again. In an embodiment of the present disclosure, the healthcare-based blockchain 108 provides for secure consent and exchange of information associated with the user data, the one or more situational parameters and the desired immunization while satisfying privacy and security regulations. The use of a blockchain is very useful as it solves the need for trust and security of health information by providing an immutable ledger where the user can access the information associated with the user data, the one or more situational parameters and the desired immunization.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 308 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

1. A computing system for facilitating management of medical and immunization history of users, the computing system comprising:
one or more hardware processors; and
a memory coupled to the one or more hardware processors, wherein the memory comprises a plurality of modules in the form of programmable instructions executable by the one or more hardware processors, and wherein the plurality of modules comprises:
a user data receiver module configured to receive user data from one or more user devices associated with a user, wherein the user data comprises: name, age, medical history, one or more medical reports, past surgical history, blood group, one or more past immunization records, one or more emergency contacts, family medical history, social history, allergies of the user, medications the user is currently taking, and medications the user have recently stopped taking;
a data detection module configured to detect one or more situational parameters associated with the user by using a location detection technique and predefined medical information upon receiving the user data, wherein the one or more situational parameters comprise a location, a place and a time of visiting a healthcare facility;
a data determination module configured to determine one or more possible immunizations to be received by the user from the healthcare facility based on the received user data, provider information and the detected one or more situational parameters, wherein the determined one or more possible immunizations are outputted on user interface screen of the one or more user devices;
an input receiver module configured to receive one or more inputs from the one or more user devices to select a desired immunization from the outputted one or more possible immunizations, wherein the desired immunization is an immunization which is one of: received and to be received by the user from the healthcare facility;
a data transmission module configured to transmit the user data, the one or more situational parameters and the desired immunization in form of a transaction to a healthcare-based blockchain via a decentralized ledger, wherein the healthcare-based blockchain includes a set of blockchain nodes, wherein the set of blockchain nodes of the healthcare-based blockchain correspond to one or more relevant authorities, and wherein the healthcare-based blockchain performs one or more blockchain operations for adding the transaction to the healthcare-based blockchain, wherein the one or more blockchain operations comprise:
verifying the received transaction upon receiving approval from the one or more relevant authorities through a consensus mechanism; and
adding the verified transaction to the healthcare-based blockchain upon successful verification of the transaction;
a request receiver module configured to receive a request from the user to access the verified transaction of the healthcare-based blockchain, wherein the verified transactions represent the user data, the one or more situational parameters and the desired immunization;
a data authentication module configured to authenticate identity of the user by using one or more authentication techniques upon receiving the request, wherein the one or more authentication techniques comprise password-based authentication, multi-factor authentication, certificate-based authentication, biometric authentication, and token-based authentication; and
a data output module configured to output the verified transaction of the healthcare-based blockchain on user interface screen of the one or more user devices upon successfully authenticating the identity of the user.

2. The computing system of claim 1, further comprising a data sharing module configured to:
receive one or more identification numbers from the one or more relevant authorities for verifying identity of the one or more relevant authorities, wherein the one or more identification numbers comprise National Provider Identifier (NPI), serial number, and lot number;
verify identity of the one or more relevant authorities by matching the one or more identification numbers with a set of prestored identification numbers;
generate one or more data-sharing parameters for sharing the user data, the one or more situational parameters and the desired immunization with the one or more relevant authorities upon verifying identity of the one or more relevant authorities, wherein the one or more data-sharing parameters comprise one or more Quick Response (QR) codes and one or more certificates; and
output the user data, the one or more situational parameters and the desired immunization on user interface screen of one or more electronic devices associated with the one or more relevant authorities by using the generated one or more data-sharing parameters.

3. The computing system of claim 1, wherein the one or more relevant authorities approve the transaction to verify the transaction by one of: providing an identification number, scanning a QR code, and selecting approve button on user interface screen of one or more electronic devices associated with the one or more relevant authorities.

4. The computing system of claim 1, further comprising an access transferring module configured to:
generate an access code for transferring the user data, the one or more situational parameters and the desired immunization to a new guardian of the user, wherein the access code is one of QR code and bar code; and
transfer the user data, the one or more situational parameters and the desired immunization from original guardian to the new guardian by using the generated access code, wherein access of the user data, the one or more situational parameters and the desired immunization is removed for the original guardian.

5. The computing system of claim 1, further comprising an immunization management module configured to:
obtain a data representative of immunization from an immunization external database, wherein the data representative of immunization comprises a list of immunizations, number of doses of each of the list of immunizations required by a person and one or more required intervals between the doses;
determine one or more immunization parameters associated with the user based on the user data, the one or more situational parameters, the desired immunization and the obtained data representative of immunization by using an immunization management-based Artificial Intelligence (AI) model, wherein the one or more immunization parameters comprise: one or more necessary immunizations, number of doses of each of the one or more necessary immunizations and time of receiving the one or more doses for each of the one or more necessary immunizations;
detect one or more providers of the one or more necessary immunizations and one or more locations of the one or more providers in vicinity of the user based on current location of the user, the determined one or more immunization parameters and predefined provider information;
plot the detected one or more locations of the detected one or more providers on a virtual map; and
output the determined one or more immunization parameters, the detected one or more providers, the one or more locations of the one or more providers and the plotted virtual map on user interface screen of the one or more user devices.

6. The computing system of claim 5, wherein the immunization management module is configured to:
obtain availability information associated with the detected one or more providers from an external provider database, wherein the availability information comprises one or more available slots for each of the one or more providers, a type of insurance plan accepted by the one or more providers, working days, and working hours of each of the one or more providers;
receive one or more inputs from the user to select a desired provider from the detected one or more providers and a desired time to visit the desired provider upon obtaining the availability information;
generate an appointment request to schedule an appointment with the desired provider at the desired time based on the obtained availability information and the received one or more inputs, wherein the appointment is scheduled with the desired provider when the generated appointment request is approved by the desired provider;
detect one or more transportation options and one or more routes to reach a location of the desired provider based on the current location of the user, the location of the desired provider and a predefined location information by using a transportation detection-based AI model, wherein the detected one or more transportation options and the detected one or more routes are outputted on user interface screen of the one or more user devices; and
receive one or more transportation inputs from the user to purchase one or more tickets of the one or more transportation options to reach the location of the desired provider upon detecting the one or more transportation options and the one or more routes, wherein the one or more transportation inputs comprise number of tickets, name of the user, a payment mode, a desired route, a desired transportation option and one or more seat numbers.

7. The computing system of claim 1, further comprising a medicine management module configured to:
obtain medicine information associated with the user from the healthcare-based blockchain, wherein the medicine information comprises one or more medications the user is currently taking;
detect a set of ingredients of the one or more medications based on the obtained medicine information and a predefined ingredient information;
receive one or more new medications for the user from one or more medical professionals;
detect a plurality of ingredients of the received one or more new medications based on the predefined ingredient information;
determine one or more possible complications corresponding to the one or more new medications based on the user data, the obtained medicine information, the detected set of ingredients, the detected plurality of ingredients and a predefined complication information by using a medication management-based AI model, wherein the one or more complications comprise slowed breathing, blood thinning, poisoning, skin rashes and increase in blood pressure; and
output the determined one or more possible complications on user interface screen of the one or more user devices and one or more provider devices associated with one or more providers.

8. The computing system of claim 7, wherein the medicine management module is configured to:
determine if the one or more new medications are similar to the one or more medications based on the detected set of ingredients of the one or more medications and the detected plurality of ingredients of the received one or more new medications by using the medication management-based AI model;
generate one or more overdosing alerts upon determining that the one or more new medications are similar to the one or more medications; and
output the generated one or more overdosing alerts on user interface screen of the one or more user devices and the one or more provider devices.

9. The computing system of claim 8, wherein the medicine management module is configured to:
obtain dose information associated with the user from at least one of: the healthcare-based blockchain, the user, a medical prescription and one or more providers, wherein the dose information comprise: one or more prescribed medications prescribed for the user, a number of required doses of the one or more prescribed medications, one or more prescribed immunizations, a number of required doses of the one or more prescribed immunizations, a required time of taking each of the required doses, a required time for performing one or more medical exercise therapies, and a time duration for which the user is required to take the one or more prescribed medications; and
generate one or more medicine alerts for the user based on the obtained dose information, wherein the one or more medicine alerts are generated for reminding the user to take the one or more prescribed medications at the required time, visit one or more medical facilities to take the one or more prescribed immunizations at the required time, stop taking the one or more prescribed medications after expiry of the time duration and perform the one or more medical exercise therapies at the required time; and
output the generated one or more medicine alerts on user interface screen of the one or more user devices.

10. The computing system of claim 1, further comprising a visit management module configured to:
obtain the provider information associated with one or more providers based on at least one of: the detected one or more situational parameters, one or more identification numbers, a provider's directory and a calendar entry of the user with the provider's name and address, wherein the provider information comprise name, address, and a medical care provided by the one or more providers, and wherein the medical care comprise injecting one or more doses of immunizations and prescribing one or more medications;
determine if the user already visited one or more medical facilities to receive a similar medical care as provided by the one or more providers based on the obtained provider information and by accessing the verified transaction of the healthcare-based blockchain;
generate one or more overdosing alerts upon determining that the user already visited the one or more medical facilities to receive the similar medical care as provided by the one or more providers; and
output the generated one or more overdosing alerts on user interface screen of the one or more user devices and one or more provider devices associated with the one or more providers.

11. A method for facilitating management of medical and immunization history of users, the method comprising:
receiving, by one or more hardware processors, user data from one or more user devices associated with a user, wherein the user data comprises: name, age, medical history, one or more medical reports, past surgical history, blood group, one or more past immunization records, one or more emergency contacts, family medical history, social history, allergies of the user, medications the user is currently taking, and medications the user have recently stopped taking;
detecting, by the one or more hardware processors, one or more situational parameters associated with the user by using a location detection technique and predefined medical information upon receiving the user data, wherein the one or more situational parameters comprise a location, a place and a time of visiting a healthcare facility;
determining, by the one or more hardware processors, one or more possible immunizations to be received by the user from the healthcare facility based on the received user data, provider information and the detected one or more situational parameters, wherein the determined one or more possible immunizations are outputted on user interface screen of the one or more user devices;
receiving, by the one or more hardware processors, one or more inputs from the one or more user devices to select a desired immunization from the outputted one or more possible immunizations, wherein the desired immunization is an immunization which is one of: received and to be received by the user from the healthcare facility;
transmitting, by the one or more hardware processors, the user data, the one or more situational parameters and the desired immunization in form of a transaction to a healthcare-based blockchain via a decentralized ledger, wherein the healthcare-based blockchain includes a set of blockchain nodes, wherein the set of blockchain nodes of the healthcare-based blockchain correspond to one or more relevant authorities, and wherein the healthcare-based blockchain performs one or more blockchain operations for adding the transaction to the healthcare-based blockchain, wherein the one or more blockchain operations comprise:
verifying the received transaction upon receiving approval from the one or more relevant authorities through a consensus mechanism;
adding the verified transaction to the healthcare-based blockchain upon successful verification of the transaction;
receiving, by the one or more hardware processors, a request from the user to access the verified transaction of the healthcare-based blockchain, wherein the verified transaction represents the user data, the one or more situational parameters and the desired immunization;
identifying, by the one or more hardware processors, identity of the user by using one or more authentication techniques upon receiving the request, wherein the one or more authentication techniques comprise password-based authentication, multi-factor authentication, certificate-based authentication, biometric authentication and token-based authentication; and
outputting, by the one or more hardware processors, the verified transaction of the healthcare-based blockchain on user interface screen of the one or more user devices upon successfully authenticating the identity of the user.

12. The method of claim 11, further comprising:
receiving one or more identification numbers from the one or more relevant authorities for verifying identity of the one or more relevant authorities, wherein the one or more identification numbers comprise National Provider Identifier (NPI), serial number, and lot number;
verifying identity of the one or more relevant authorities by matching the one or more identification numbers with a set of prestored identification numbers;
generating one or more data-sharing parameters for sharing the user data, the one or more situational parameters and the desired immunization with the one or more relevant authorities upon verifying identity of the one or more relevant authorities, wherein the one or more data-sharing parameters comprise one or more Quick Response (QR) codes and one or more certificates; and
outputting the user data, the one or more situational parameters and the desired immunization on user interface screen of one or more electronic devices associated with the one or more relevant authorities by using the generated one or more data-sharing parameters.

13. The method of claim 11, wherein the one or more relevant authorities approve the transaction to verify the transaction by one of: providing an identification number, scanning a QR code, and selecting approve button on user interface screen of one or more electronic devices associated with the one or more relevant authorities.

14. The method of claim 11, further comprising:
generating an access code for transferring the user data, the one or more situational parameters and the desired immunization to a new guardian of the user, wherein the access code is one of QR code and bar code; and
transferring the user data, the one or more situational parameters and the desired immunization from original guardian to the new guardian by using the generated access code, wherein access of the user data, the one or more situational parameters and the desired immunization is removed for the original guardian.

15. The method of claim 11, further comprising:
obtaining a data representative of immunization from an immunization external database, wherein the data representative of immunization comprises a list of immunizations, number of doses of each of the list of immunizations required by a period and one or more required intervals between the doses;
determining one or more immunization parameters associated with the user based on the user data, the one or more situational parameters, the desired immunization and the obtained data representative of immunization by using an immunization management-based Artificial Intelligence (AI) model, wherein the one or more immunization parameters comprise: one or more necessary immunizations, number of doses of each of the one or more necessary immunizations and time of receiving the one or more doses for each of the one or more necessary immunizations;
detecting one or more providers of the one or more necessary immunizations and one or more locations of the one or more providers in vicinity of the user based on current location of the user, the determined one or more immunization parameters and predefined provider information;
plotting the detected one or more locations of the detected one or more providers on a virtual map; and
outputting the determined one or more immunization parameters, the detected one or more providers, the one or more locations of the one or more providers and the plotted virtual map on user interface screen of the one or more user devices.

16. The method of claim 15, further comprising:
obtaining availability information associated with the detected one or more providers from an external provider database, wherein the availability information comprises one or more available slots for each of the one or more providers, a type of insurance plan accepted by the one or more providers, working days and working hours of each of the one or more providers;
receiving one or more inputs from the user to select a desired provider from the detected one or more providers and a desired time to visit the desired provider upon obtaining the availability information;
generating an appointment request to schedule an appointment with the desired provider at the desired time based on the obtained availability information and the received one or more inputs, wherein the appointment is scheduled with the desired provider when the generated appointment request is approved by the desired provider;
detecting one or more transportation options and one or more routes to reach a location of the desired provider based on the current location of the user, the location of the desired provider and a predefined location information by using a transportation detection-based AI model, wherein the detected one or more transportation options and the detected one or more routes are outputted on user interface screen of the one or more user devices; and
receiving one or more transportation inputs from the user to purchase one or more tickets of the one or more transportation options to reach the location of the desired provider upon detecting the one or more transportation options and the one or more routes, wherein the one or more transportation inputs comprise number of tickets, name of the user, a payment mode, a desired route, a desired transportation option and one or more seat numbers.

17. The method of claim 11, further comprising:
obtain medicine information associated with the user from the healthcare-based blockchain, wherein the medicine information comprises one or more medications the user is currently taking;
detecting a set of ingredients of the one or more medications based on the obtained medicine information and a predefined ingredient information;
receiving one or more new medications for the user from one or more medical professionals;
detecting a plurality of ingredients of the received one or more new medications based on the predefined ingredient information;
determining one or more possible complications corresponding to the one or more new medications based on the user data, the obtained medicine information, the detected set of ingredients, the detected plurality of ingredients and a predefined complication information by using a medication management-based AI model, wherein the one or more complications comprise slowed breathing, blood thinning, poisoning, skin rashes and increase in blood pressure; and
outputting the determined one or more possible complications on user interface screen of the one or more user devices and one or more provider devices associated with one or more providers.

18. The method of claim 17, further comprising:
determining if the one or more new medications are similar to the one or more medications based on the detected set of ingredients of the one or more medications and the detected plurality of ingredients of the received one or more new medications by using the medication management-based AI model;
generating one or more overdosing alerts upon determining that the one or more new medications are similar to the one or more medications; and
outputting the generated one or more overdosing alerts on user interface screen of the one or more user devices and the one or more provider devices.

19. The method of claim 11, further comprising:
obtaining dose information associated with the user from at least one of: the healthcare-based blockchain, the user, a medical prescription and one or more providers, wherein the dose information comprise: one or more prescribed medications prescribed for the user, a number of required doses of the one or more prescribed medications, one or more prescribed immunizations, a number of required doses of the one or more prescribed immunizations, a required time of taking each of the required doses, a required time for performing one or more medical exercise therapies, and a time duration for which the user is required to take the one or more prescribed medications; and
generating one or more medicine alerts for the user based on the obtained dose information, wherein the one or more medicine alerts are generated for reminding the user to take the one or more prescribed medications at the required time, visit one or more medical facilities to take the one or more prescribed immunizations at the required time, stop taking the one or more prescribed medications after expiry of the time duration and perform the one or more medical exercise therapies at the required time; and
outputting the generated one or more medicine alerts on user interface screen of the one or more user devices.

20. The method of claim 11, further comprising:
obtaining the provider information associated with one or more providers based on at least one of: the detected one or more situational parameters, one or more identification numbers, a provider's directory and a calendar entry of the user with the provider's name and address, wherein the provider information comprise name, address, and a medical care provided by the one or more providers, and wherein the medical care comprise injecting one or more doses of immunizations and prescribing one or more medications;
determining if the user already visited one or more medical facilities to receive a similar medical care as provided by the one or more providers based on the obtained provider information and by accessing the verified transaction of the healthcare-based blockchain;
generating one or more overdosing alerts upon determining that the user already visited the one or more medical facilities to receive the similar medical care as provided by the one or more providers; and
outputting the generated one or more overdosing alerts on user interface screen of the one or more user devices and one or more provider devices associated with the one or more providers.
